(19) United States
(12) Patent Application Publication
(10) Pub. No.: US 2009/0299476 A1
(43) Pub. Date: Dec. 3, 2009

(54) TISSUE PROSTHESIS

(76) Inventors: Ashish Diwan, New South Wales (AU); Zoran Milijasevic, New South Wales (AU)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/300,113

(22) PCT Filed: May 19, 2006
(86) PCT No.: PCT/AU07/00679

§ 371 Date: Apr. 21, 2009

Related U.S. Application Data
(60) Provisional application No. 60/747,722, filed on May 19, 2006.

(51) Int. Cl. A61F 2/44

(52) U.S. Cl. 623/17.12; 623/23.72

(57) ABSTRACT

A tissue prosthesis system includes an envelope (10) which is able substantially to conform to a shape of a cavity (30) formed in a site in a patient's body, the envelope (10) having a filler opening (16) to enable access to be gained to an interior of an envelope (10). A filler mechanism (12) is connectable to the opening (16) of the envelope (10). A filler material (32) is chargeable into the interior of the envelope (10) via the filler mechanism (12) to cause expansion of the envelope (10) substantially to conform to the shape of the cavity (30). A separation arrangement is associated with the envelope (10) and the filler material (12) to facilitate separation of the envelope (10) from the filler material (12), in use.

### CROSS-REFERENCE TO RELATED APPLICATIONS

The present application claims priority from U.S. Provisional Patent Application No 60/747,722 filed on 19 May 2006, the contents of which are incorporated herein by reference.

### FIELD

This invention relates, generally, to a tissue prosthesis and, more particularly but not necessarily exclusively, to an intervertebral disc prosthesis.

### BACKGROUND

Various techniques have been proposed for dealing with age or injury related intervertebral disc degeneration. Two techniques in use are disc removal and fusion. Both of these techniques involve major invasive surgery with the related risks. More recently, another technique employed has involved the replacing of a degenerative disc with an artificial disc. This, once again, is carried out using major invasive techniques.

An intervertebral disc comprises two parts, an annulus fibrosis surrounding a nucleus pulposus. The intervertebral disc cooperates with end plates of the vertebrae between which it is sandwiched. Still more recently, techniques have been proposed to replace only the nucleus pulposus of the disc in circumstances where the degeneration is detected at a sufficiently early stage. Such techniques may be able to be performed in a minimally invasive manner.

Ideally, the fewer materials implanted into a body, the better as the likelihood of rejection is reduced.

### SUMMARY

According to a first aspect of the invention, there is provided a tissue prosthesis system which includes:

an envelope which is able substantially to conform to a shape of a cavity formed in a site in a patient's body, the envelope having a filler opening to enable access to be gained to an interior of an envelope;

a filler mechanism connectable to the opening of the envelope;

a filler material chargeable into the interior of the envelope via the filler mechanism to cause expansion of the envelope substantially to conform to the shape of the cavity; and

a separation arrangement associated with the envelope and the filler material to facilitate separation of the envelope from the filler material, in use.

The separation mechanism may be selected from the group consisting of a chemical separation mechanism, a mechanical separation mechanism and a thermal separation mechanism.

In one embodiment, the separation mechanism may be a chemical separation mechanism which relies on chemical or ionic forces between the envelope and the filler material. In another embodiment, the separation mechanism may be a chemical separation mechanism which comprises a non-reacting leechant between an inner surface of the envelope and the filler material for effecting separation of the envelope from the filler material. In still another embodiment, the separation mechanism may be a chemical separation mechanism which relies on swelling, weakening or dissolving of the envelope.

The separation mechanism may be a mechanical separation mechanism, the envelope and the filler material being of different materials.

The separation mechanism may be a thermal separation mechanism which relies on achieving a thermal differential between the envelope and the filler material. The achieving of a thermal differential may be effected by a heating coil in one of the filler material and the envelope. Instead, the thermal separation may occur as a result of one of an exothermic reaction and an endothermic reaction resulting from the curing of the filler material.

At least one rupture zone may be defined in the envelope, the rupture zone rupturing upon application of a predetermined force to effect removal of the envelope from the filler material.

The filler opening may be a closable filler opening. The filler opening may be closed by a non-return filler valve.

The envelope may be of a re-absorbable material.

According to a second aspect of the invention, there is provided a method of forming a tissue prosthesis in situ, the method comprising:

inserting an envelope into a cavity at a site in a patient's body, the envelope being able substantially to conform to a shape of the cavity;

charging a filler material into an interior of the envelope to cause expansion of the envelope to conform substantially to the shape of the cavity;

allowing the filler material to set at least partially; and

causing separation of the envelope from the filler material.

The method may include withdrawing the separated envelope from the at least partially set filler material.

Prior to inserting the envelope into the cavity, the method may include forming the cavity by removing degenerative tissue at the site. The method may include accessing the site by inserting an introducer through an aperture formed in tissue surrounding the site and removing degenerative tissue from the site.

The method may include attaching the envelope to a distal end of a tubular filler mechanism and inserting the delivery device through the aperture so that the envelope is within the cavity at the site. Thus, the method may include charging the filler material into the interior of the envelope via the tubular filler mechanism.

The method may include causing separation of the envelope from the filler material by at least one of chemical separation, mechanical separation and thermal separation.

In one embodiment, the method may include chemically separating the envelope from the filler material by reliance on molecular repulsion forces between the filler material and the envelope. In another embodiment, the method may include chemically separating the envelope from the filler material by use of a non-reacting leechant between the filler material and the envelope. The leechant may be generated between the envelope and the filler material or it may be pre-applied to an interior surface of the envelope. Thus, the method may include causing separation of the envelope from the filler material by the existence of a pre-formed boundary layer between the envelope and the filler material.

The method may include causing separation of the envelope from the filler material by at least one of swelling, weakening or dissolving the envelope.

The method may include creating a thermal differential between the envelope and the filler material to cause separation of the envelope from the filler material. The method may include creating the thermal differential by the application of localised heating to one of the envelope and the filler material. The application of localised heating may be achieved by, for example, the use of a heating coil in one of the envelope and the filler material. Instead, the thermal differential may occur as a result of one of an exothermic reaction and an endothermic reaction resulting from the curing of the filler material.

The method may further include withdrawing of the envelope by rupturing at least one zone of weakness in the envelope. The method may include causing rupturing of the envelope by over-inflating the envelope using the filler material.

Instead of withdrawing the separated envelope, the method may include, after separating the envelope from the filler material, causing the envelope to be re-absorbed.

According to a third aspect of the invention, there is provided a component for a tissue prosthesis, the component including:

an envelope which is able substantially to conform to a shape of a cavity formed at a site in a patient's body, the envelope defining a filler opening via which a filler material is charged into an interior of the envelope; and

at least one rupture zone arranged on the envelope for facilitating rupturing of the envelope.

The envelope may include a separation mechanism carried by the envelope to facilitate separation of the envelope from the filler material, in use. The separation mechanism may be selected from the group consisting of a chemical separation mechanism, a mechanical separation mechanism and a thermal separation mechanism.

The envelope may be of an elastomeric material capable of expanding up to 10 times its relaxed state.

The at least one rupture zone may comprise a line of weakness formed in the envelope.

The filler opening may be a closable filler opening. The filler opening may be closed by a non-return filler valve.

The envelope may be of a re-absorbable material.

### BRIEF DESCRIPTION OF DRAWINGS

FIG. 1 shows a schematic side view of an initial step in the formation of a tissue prosthesis, in accordance with an embodiment of the invention, at a site in a patient's body;

FIG. 2 shows a schematic side view of a following step in the formation of the prosthesis;

FIG. 3 shows a schematic side view of a further step in the formation of the prosthesis;

FIG. 4 shows a schematic side view of yet a further step in the formation of the prosthesis; and

FIG. 5 shows a schematic side view of the prosthesis in position.

### DETAILED DESCRIPTION OF EXEMPLARY EMBODIMENT

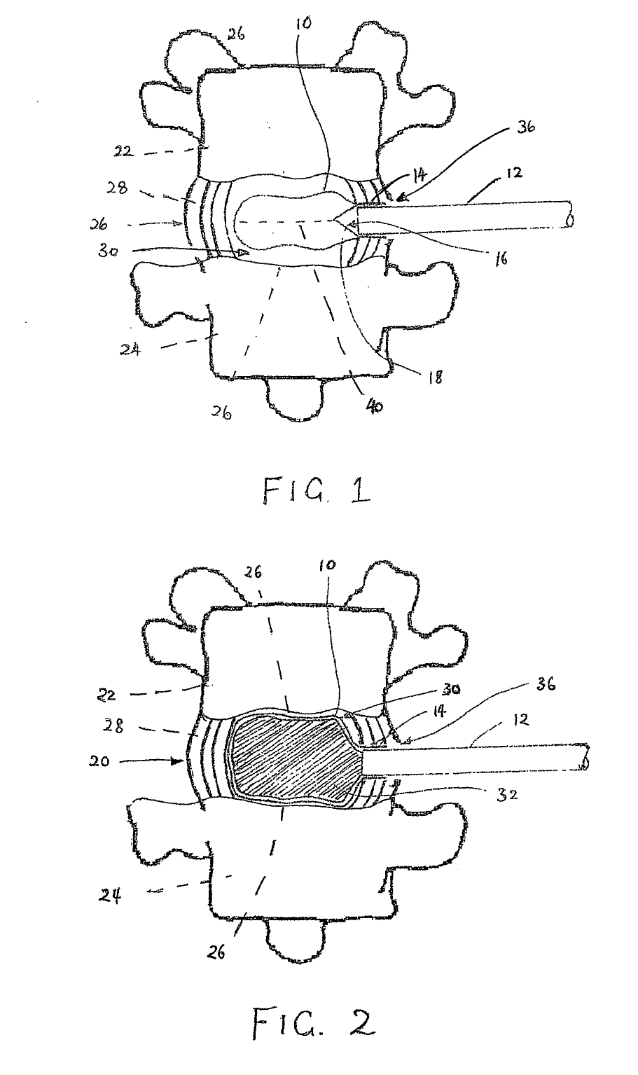

FIGS. 1-5 show various stages in a method of forming a tissue prosthesis in situ, in accordance with an embodiment of the invention.

The method involves, initially, attaching an envelope 10 to the end of a filler mechanism in the form of a delivery device 12. For this purpose, the envelope has a neck portion 14 within which a distal end of the delivery device 12 is receivable. Further, the envelope 10 has a closable opening 16 which is closed off by a non-return valve 18 such as, for example, a duckbill valve.

While the invention is applicable in numerous applications where soft tissue is to be replaced, the invention is particularly applicable in replacing a nucleus pulposus of an intervertebral disc indicated schematically at 20. An embodiment of the invention will be described with reference to that application below.

The disc 20 is located between adjacent vertebrae 22 and 24. End plates 26 of the vertebrae 22 and 24 and an annulus fibrosis 28 of the disc 20 define a cavity 30 in which the envelope 10 is receivable in a deflated condition.

A tissue prosthesis system, also in accordance with an embodiment of the invention, comprises a filler material 32 (FIG. 2) which, in use, is charged into an interior 34 of the deflated envelope 10 to cause expansion of the envelope 10.

In use, to replace a degenerative nucleus pulposus of the disc 20, access is gained to an interior of the disc 20 through an opening 36 made in the annulus fibrosis 28 of the disc 20 using a trocar (not shown) or some other similar device received through an introducer (also not shown). Access is gained to the disc 20 in a minimally invasive manner through an incision made in the skin of the patient.

Once the interior of the disc 20 has been accessed, the degenerative nucleus pulposus of the disc can, optionally, be removed by performing a nucleotomy. This can be done in one of a number of ways, for example, mechanically, chemically, thermally, or the like. The device which performs the nucleotomy is inserted into the disc 20 through the access opening 36 via the introducer after removal of the trocar. In some instances, the nucleus pulposus of the disc 20 may have degenerated to such an extent that a cavity 30 which is sufficiently large to accommodate the prosthesis already exists and it is not necessary to perform a nucleotomy.

Whether or not a nucleotomy is performed, the absence of nuclear material results in the cavity 30 being present. The envelope 10, in its deflated condition, is inserted into the cavity 30 of the disc 20 by means of the delivery device 12 through the introducer. The filler material 32 is charged into the interior 34 of the envelope 10 to cause expansion of the envelope 10 until the envelope 10 conforms substantially to the shape of the cavity 30. The non-return valve 18 inhibits backflow of the filler material 32 up the filler device 12.

Typically, the envelope 10 is of an expansible material. In a preferred embodiment, the envelope 10 is an elastomeric material, such as a silicone material. It will, however, be appreciated that the envelope 10 could, instead, be non-elastic.

The filler material 32 is, optionally, of the same material as the envelope 10. Thus, in the case of a silicone envelope 10, the filler material 32 may be of a silicone material as well. However, as will be described in greater detail below, there is no need to obtain homogeneity between the envelope 10 and the filler material 32 and, as a result, the filler material 32 could be a different material or a different grade of material to that of the envelope 10.

The filler material 32 is of a type which flows in a pre-mixed configuration. The filler material 32 is mixed together upstream of the delivery device 12 and mixes in the delivery device 12 and in the interior 34 of the envelope 10. The mixed material cures or sets to a desired consistency which assists in restoring the bio-mechanical properties of the disc 20 at least to some extent.

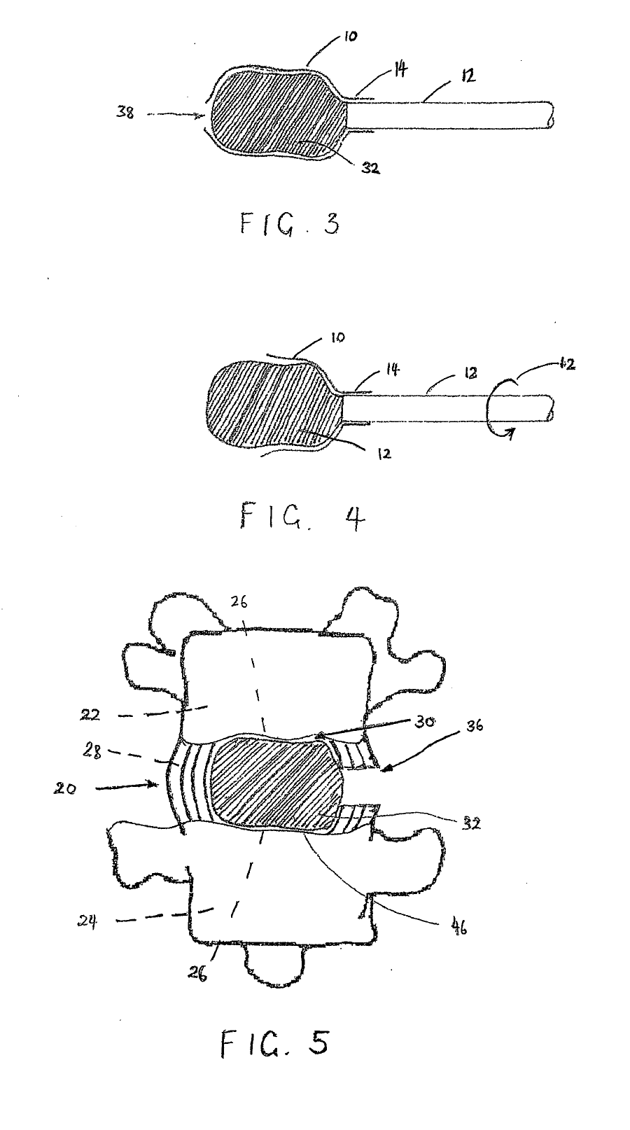

The system in accordance with an embodiment of the invention makes use of an envelope 10 which separates from the filler material 32 and then, optionally, ruptures. Thus, as shown in FIGS. 3 of the drawings, after the filler material 32 has cured or set within the envelope 10, the envelope 10 separates from the filler material 32 and ruptures as shown, schematically, at 38 in FIG. 3 of the drawings.

The manner in which the envelope 10 separates can be achieved in one of a number of ways. Firstly, a chemical reaction could occur between the envelope 10 and the filler material 32 which causes repulsion of the envelope 10 from the filler material 32 resulting in separation of the envelope 10 from the filler material 32.

Instead, one of the envelope 10 and the filler material 32 leeches a non-reacting material which results in the generation of a barrier layer which reacts with the envelope 10 causing separation of the envelope 10 from the filler material 32. The leechant, or leeched material, is an uncrosslinked monomer, a polymer or any other volatile substance which is available to effect separation between the envelope 10 and the filler material 32. In this case, for example, the filler material 32 is a silicone material which is cured at a sufficiently slow rate to allow some material to leech out or be exuded as an oil. This oil stops bonding between the filler material 32 and the envelope 10 and acts as a lubricant between the filler material 32 and the envelope 10. In addition, the oil is absorbed by the envelope 10. In the case of a silicone envelope, the envelope 10 swells as it absorbs the oil. Swelling of the silicone envelope 10 causes weakening of the envelope 10 facilitating its rupturing.

In another variation, the boundary layer could, instead, be pre-applied as a coating to an interior surface of the envelope 10, prior to insertion of the envelope 10 into the disc 20. This coating either reacts with the filler material 32 to cause separation of the envelope 10 from the filler material 32 or, instead, the presence of the coating on its own, i.e. without the need to react, could maintain separation between the filler material 32 and the envelope 10. The coating is in the form of mineral oil, silicone oil or any other suitable substance which forms a physical barrier between the filler material 32 and the envelope 10.

In still another embodiment, the envelope 10 is separated from the filler material 32 by a chemical separation mechanism which relies on swelling, weakening or dissolving of the envelope. The leechant or coating is absorbed by the envelope 10 resulting in weakening of the envelope 10 and its effecting the separation of the envelope 10 from the filler material. For example, in the case of a silicone envelope 10 and silicone filler material 32, the filler material 32 leeches out a leechant as it cures and the envelope 10 absorbs the leechant causing it to swell, separate from the filler material 32 and weaken. The filler material 32 may give off acetic acid as it cures with the envelope 10 being susceptible to dissolution from the acetic acid given off by the filler material 32.

In the case where the envelope 10 and the filler material 32 are of different materials, separation may be effected between the filler material 32 and the envelope 10 on curing of the filler material 32 after which the envelope 10 separates from the filler material 32 and ruptures, for example, along a zone of weakness 40. For example the filler material 32 may be of a polyurethane filler material while the envelope 10 is of a silicone material or vice versa. The filler material 32 and the envelope 10 could also be of different grades of silicone material.

Still further, separation of the envelope 10 from the filler material 32 may be achieved by thermal means. This may be achieved by having an electrical coil in one of the envelope 10 and the filler material 32 to create a temperature differential between the envelope 10 and the filler material 32. In so doing, separation of the envelope 10 from the filler material 32 is achieved. Thereafter, rupturing of the envelope 10 allows the envelope 10 to be withdrawn.

The rupturing of the envelope 10 can be achieved in one of a number of ways. For example, the envelope 10 could be over-inflated with filler material 32 causing rupturing of the envelope 10. Instead, the envelope 10 may have lines of weakness such as, for example, shown at 40 in FIG. 1 of the drawings along which the envelope 10 can rupture when filled with the filler material 32.

Regardless of the manner in which the envelope 10 separates and ruptures, after rupturing, the envelope 10 puckers in a proximal direction as shown in FIG. 4 of the drawings. Rotation of the delivery device 12 about an arrow 42 may enhance this withdrawal of the envelope 10 in a proximal direction facilitating its withdrawal from the cured filler material 32.

FIG. 5 shows a completed prosthesis 46, consisting essentially of the filler material 32, conforming to the shape of the cavity 30 of the disc 20 after withdrawal of the envelope 10 from around the filler material 32. The filler material 32, itself, may occlude the opening 36 or, instead, a plug of some sort may be inserted into the opening 36 to retain the filler material 32 within the disc 20.

Instead of the envelope 10 rupturing, the envelope 10 could be of a material which is re-absorbable by the body. Thus, after separation of the envelope 10 from the filler material 32, the envelope 10 is left within the disc 20 for subsequent re-absorption. Possible re-absorbable materials which could be used for the envelope 10 include polyesters (such as polyglycolic acid, polylactic acid, polycaprolactone), polyanhydrides, or the like.

It is an advantage of the invention that a system is provided which does not necessitate that the envelope 10 and the filler material 32 are of the same material to result in a homogeneous prosthesis. Thus, the envelope 10 could be of a first material with the filler material 32 being of a second, different material having the required viscosity and settable properties. The filler material 32, after removal of the envelope 10, functions as the nuclear prosthesis 46. It is a further advantage of the invention that, by removing the envelope 10, there is no interface and a homogeneous prosthesis 46 is indeed achieved. Further, by removing the envelope 10, there is no neck portion 14 protruding through the annulus fibrosis 28 of the disc 20. It will be appreciated that the neck portion 14 of the envelope may be perceived by the body as a foreign object and may lead to issues of localised irritation or, even, rejection.

It is still a further advantage of the invention that a system is provided which uses the envelope 10 to contain the filler material 32 while the filler material 32 cures or sets after which the envelope 10 is removed. The envelope 10 therefore contains the filler material 32 while it sets inhibiting leakage into undesirable areas such as annular fissures of the disc 20 and to inhibit any leechants from the filler material 32 reacting with the disc 20. The envelope 10 can also dictate the shape of the filler material 32 and, consequently, the shape of the final prosthesis 46. After setting of the filler material 32, the envelope 10 is removed resulting in a reduced number of possibly different materials or compounds remaining in the patient's body.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A tissue prosthesis system which includes:
an envelope which is able substantially to conform to a shape of a cavity formed in a site in a patient's body, the envelope having a filler opening to enable access to be gained to an interior of an envelope;
a filler mechanism connectable to the opening of the envelope;
a filler material chargeable into the interior of the envelope via the filler mechanism to cause expansion of the envelope substantially to conform to the shape of the cavity; and
a separation arrangement associated with the envelope and the filler material to facilitate separation of the envelope from the filler material, in use.

2. The system of claim 1 in which the separation mechanism is selected from the group consisting of a chemical separation mechanism, a mechanical separation mechanism and a thermal separation mechanism.

3. The system of claim 2 in which the separation mechanism is a chemical separation mechanism which relies on chemical or ionic forces between the envelope and the filler material.

4. The system of claim 2 in which the separation mechanism is a chemical separation mechanism which comprises a non-reacting leechant between an inner surface of the envelope and the filler material for effecting separation of the envelope from the filler material.

5. The system of claim 2 in which the separation mechanism is a chemical separation mechanism which relies on swelling, weakening or dissolving of the envelope.

6. The system of claim 2 in which the separation mechanism is a mechanical separation mechanism, the envelope and the filler material being of different materials.

7. The system of claim 2 in which the separation mechanism is a thermal separation mechanism which relies on achieving a thermal differential between the envelope and the filler material.

8. The system of claim 7 in which the achieving of a thermal differential is effected by a heating coil in one of the filler material and the envelope.

9. The system of claim 7 in which the thermal separation occurs as a result of one of an exothermic reaction and an endothermic reaction resulting from the curing of the filler material.

10. The system of claim 1 which comprises at least one rupture zone defined in the envelope, the rupture zone rupturing upon application of a predetermined force to effect removal of the envelope from the filler material.

11. The system of claim 1 in which the filler opening is a closable filler opening.

12. The system of claim 11 in which the filler opening is closed by a non-return filler valve.

13. The system of claim 1 in which the envelope is of a re-absorbable material.

14. A method of forming a tissue prosthesis in situ, the method comprising:
inserting an envelope into a cavity at a site in a patient's body, the envelope being able substantially to conform to a shape of the cavity;
charging a filler material into an interior of the envelope to cause expansion of the envelope to conform substantially to the shape of the cavity;
allowing the filler material to set at least partially; and
causing separation of the envelope from the filler material.

15. The method of claim 14 which includes withdrawing the separated envelope from the at least partially set filler material.

16. The method of claim 14 which includes, prior to inserting the envelope into the cavity, forming the cavity by removing degenerative tissue at the site.

17. The method of claim 16 which includes accessing the site by inserting an introducer through an aperture formed in tissue surrounding the site and removing degenerative tissue from the site.

18. The method of claim 14 which includes attaching the envelope to a distal end of a tubular filler mechanism and inserting the delivery device through the aperture so that the envelope is within the cavity at the site.

19. The method of claim 18 which includes charging the filler material into the interior of the envelope via the tubular filler mechanism.

20. The method of claim 14 which includes causing separation of the envelope from the filler material by at least one of chemical separation, mechanical separation and thermal separation.

21. The method of claim 20 which includes chemically separating the envelope from the filler material by reliance on molecular repulsion forces between the filler material and the envelope.

22. The method of claim 20 which includes chemically separating the envelope from the filler material by use of a non-reacting leechant between the filler material and the envelope.

23. The method of claim 22 which includes causing separation of the envelope from the filler material by the existence of a pre-formed barrier between the envelope and filler material.

24. The method of claim 23 which includes causing separation of the envelope from the filler material by at least one of swelling, weakening and dissolving the envelope.

25. The method of claim 20 which includes creating a thermal differential between the envelope and the filler material to cause separation of the envelope from the filler material.

26. The method of claim 25 which includes creating the thermal differential by the application of localised heating to one of the envelope and the filler material.

27. The method of claim 25 which includes causing the thermal differential to occur as a result of one of an exothermic reaction and an endothermic reaction resulting from the curing of the filler material.

28. The method of claim 14 which includes withdrawing of the envelope by rupturing at least one zone of weakness in the envelope.

29. The method of claim 27 which includes causing rupturing of the envelope by over-inflating the envelope using the filler material.

30. The method of claim 14 which includes, after separating the envelope from the filler material, causing the envelope to be re-absorbed.

31. A component for a tissue prosthesis, the component including:
an envelope which is able substantially to conform to a shape of a cavity formed at a site in a patient's body, the envelope defining a filler opening via which a filler material is charged into an interior of the envelope; and
at least one rupture zone arranged on the envelope for facilitating rupturing of the envelope.

32. The component of claim 31 which includes a separation mechanism carried by the envelope to facilitate separation of the envelope from the filler material, in use.

33. The component of claim 32 in which the separation mechanism is selected from the group consisting of a chemical separation mechanism, a mechanical separation mechanism and a thermal separation mechanism.

34. The component of claim 31 in which the envelope is of an elastomeric material capable of expanding up to 10 times its relaxed state.

35. The component of claim 31 in which the at least one rupture zone comprises a line of weakness formed in the envelope.

36. The component of claim 31 in which the filler opening is a closable filler opening.

37. The component of claim 36 in which the filler opening is closed by a non-return filler valve.

38. The component of claim 31 in which the envelope is of a re-absorbable material.
